(19) United States
(12) Patent Application Publication
(10) Pub. No.: US 2021/0000867 A1
(43) Pub. Date: Jan. 7, 2021

(54) NEW ROUTE AND ENGINEERING PROTOCOLS FOR ALZHEIMERS DISEASE PREVENTION, TREATMENT AND NEURONAL REBUILDING

(71) Applicant: Glycan Industries LLC, Pasadena, CA (US)

(72) Inventor: Christian Daniel Assoun, Geneva (CH)

(73) Assignee: Glycan Industries LLC, Pasadena, CA (US)

(21) Appl. No.: 16/906,450

(22) Filed: Jun. 19, 2020

Related U.S. Application Data
(60) Provisional application No. 62/878,558, filed on Jul. 25, 2019.

(30) Foreign Application Priority Data
Jun. 19, 2019 (LU) LU101272
Apr. 7, 2020 (IE) S2020/0056

(51) Int. Cl. A61K 33/30; A61K 31/695; A61K 47/54; A61K 47/40; A61K 9/00; A61K 31/575; A61K 35/28; A61N 5/06; A61B 5/055; G01R 33/56; G01R 33/44

(52) U.S. Cl. CPC A61K 33/30; A61K 31/695; A61K 47/549; A61K 47/40; A61K 9/0056; A61N 2005/0657; A61K 35/28; A61N 5/062; A61B 5/055; G01R 33/5601; G01R 33/445; A61K 31/575

(57) ABSTRACT

Compositions comprising zinc Zn-FLNT4 and silicon SiHR57 molecules and protocols for their delivery to persons are disclosed, for mitigating and remediating neurodegenerative and cardiovascular diseases. The compositions and protocols are based upon the dissolution properties of neurotoxic compounds accumulated in brain tissues, the use of magnetic sources generating external electromagnetic radio waves or fields and the use of quantum tools such as nMRi and MHD-MGD to incentivise molecular flows.

### CROSS-REFERENCE TO RELATED APPLICATIONS

This application claims the benefit of priority to Luxembourg patent Application No. LU101272, filed 19 Jun. 2019, U.S. Provisional 62/878,558, filed 25 Jul. 2019, and Ireland patent Application No. S2020/0056, filed 7 Apr. 2020, each of which are hereby incorporated by reference in their entirety.

### FIELD OF INVENTION

The present invention relates to compositions and their use in methods for the prevention or treatment of pathologies, including neurodegenerative, cardiovascular diseases and different stages of coma.

### BACKGROUND TO INVENTION

Many neurodegenerative and cardiovascular pathologies are today still considered to be irreversible, such as Alzheimer's Disease (‘AD’), Parkinson's Disease, Creutzfeld-Jakob's Disease (fCJD), different types and stages of coma, cardiovascular accidents (‘CVA’) including strokes, diabetes type III involving resistance to insulin in the brain, and neuro-immunodeficiency (leukocytes trafficking) in the brain-blood barrier (‘BBB’), which is the regulating seat of neuro-dynamics fluxes.

CVA diseases are broadly associated with a molecular pathology of cholesterol (‘CHO’), which is illustrated in FIG. 1, and the intermediate stages of which involve the accumulation and polymerisation of monomer-like atheromatous plaques. These plaques induce logical disorders in the kinetics of blood flow and cause the loss of integrity of the BBB, eventually conducive of a CVA stroke associated with total or partial blockage of brain activity and memory capacity. The major protein component of these plaques is beta amyloid peptide Aβ.

The cholesterol molecule comprises four carbon rings denoted A, B, C and D (cyclopentano-perhydro-phenanthrene ring), five methyl CH₃ radicals and eight asymmetric carbons (carbons 3, 8, 9, 10, 13, 14, 17 and 20), which makes 28 or 256 stereoisomers of which only one exists: the levorotatory 3β-ol. Cholesterol has a hydroxyl group —OH on carbon 3 (C3). This group is the polar head and therefore the hydrophilic part of cholesterol. The —OH function of cholesterol low-density lipoprotein (‘LDL’) can be esterified with a fatty acid which renders the molecule substantially insoluble in water or blood and will lead to toxic plaques.

Esterification of sterols, including CHO LDL, is a collateral effect which is associated to the co-polymerization of cholesterol: the monomer of cholesterol (‘CHO’) can adopt a self-assembling configuration and a polymeric-like structure under UV radiation, derived from membrane cells proton H⁺ velocities. In the case of esterification, CHO is insoluble in haemoglobin (‘Hb’) and blood flow, and this property seems to favour fibrillogenesis, tangles and plaques mainly in blood, arteries, the brain and the BBB.

We can consider CHO LDL as prominent co-factor of Reactive Oxygen Species (‘ROS’) and DNA alkylation mainly due to the presence of these five CH₃ methyl radicals, conducive of DNA damage and possible losses of telomere integrity. This fact might provoke prominent disorders at cellular level and generate by-products as prions, fragments, Tau proteins and promoters of Aβ plaques in neuronal tissue and whole brain areas, generally illustrated in FIGS. 2, 3 and 4.

Bacterial agents and exogenous contaminants such as pesticides, nitrites, nitrates, dioxins-furans-PCBs-preservatives (endocrine disruptors), fluoro-aerosols (industries), insufficiently bio-degraded pharmaceutical molecules (hormones and parts of hormones-antibiotics from animal breeding), besides heavy and toxic metals (Aluminium Al³⁺ and other toxic metals (including Hg², Mercury, especially in the form of Mono Methyl Mercury (‘MMM’)) all constitute molecular or elementary vectors (metals-atoms-elements) that are important and aggravating sources of oxidative stress of Reactive Oxygen Species (‘ROS’), which inhibit the performance of synaptic cleft (neuro-mediators) and contribute to develop neurodegenerative pathologies, in some individuals quicker than in others according to their respective genetic potentials. An important point of ROS, oxidative stress is the formation of highly oxidative pathogens such as H₂O₂ or hydrogen peroxide and/or assimilated.

This phenomenon appears comparable to the increase of the entropy of the medium, which exists because of the relative duration of the integrity biological variable depending on the immune and genetic resistance and telomere depletion of individuals.

Pathologic cholesterol LDL can thus be considered as oxidative and mutagenetic and, as an agent of carcinogenesis and immunodeficiency, heavily responsible for neuropathic diseases among others pathologies, including hepatic disorders.

Amongst neuropathic diseases, Alzheimer's Disease is a multifactorial pathology characterized by the formation of extracellular plaques integrated by the β-amyloid peptide (Aβ), the intracellular neurofibrillary tangles of the hyperphosphorylated tau protein and the presence of chronic neuroinflammation that causes progressive deterioration of memory and cognitive functions. Current therapeutic strategies to improve memory deficit aim at preventing the formation and accumulation of Aβ and the tau protein phosphorylation (Hampel et al., 2018; Hu et al., 2019). Beyond these targets, other common facts of the AD pathophysiology, such as oxidative damage and inflammation, may also be therapeutic objectives in order to slow down the memory loss progression (Salomone et al., 2012). The multifactorial origin of AD limits the design of effective drugs for AD prevention and/or treatment.

Thus, due to the complex interrelation between the different pathophysiological mechanisms involved in this disease, the use of therapies selectively addressed to reduce or block one possible cause of the disease does not seem to be adequate to reverse neuronal degeneration. On the contrary, pharmacological therapies addressed to act on all or most factors recognized today as responsible for the disease constitute a more effective a priori approach for the design of new generation drugs. These new strategies are known as “multi-target therapies” (Hopkins, et al., 2008). Thus, at present multi-target drugs for AD have been designed to promote inhibitory effects on the acetylcholinesterase (AChE) or ButyrylCholinesterase (BuChE) activities together with other relevant actions such as β-secretase inhibition, βA peptide anti-aggregation, improving the antioxidant status, acting as antihistaminic and/or CB1 cannabinoid receptor antagonist, or metal chelator. In addition, other drugs have been designed to con-joint β-secretase inhibitory activity with metal chelating capacity effects or to obtain drugs modulating both γ-secretase activity and PPARγ receptors (Connolly et al. 2018; Hu et al. 2019; Ibrahim and Gabr, 2019; Tamagno et al., 2008). In recent years, emergent evidences support the implication of the innate immunity, particularly of microglia in the pathogenesis of AD (Heneka et al., 2015a). The protein aggregates deposited in AD brains are recognized by the microglia cell receptors and, consequently, triggering an innate immunity response, characterized by the release of several inflammatory mediators, who contribute to the progression of the disease (Heneka et al., 2015b). In addition, recent genetic studies have shown that several genes related to innate immunity are associated with the risk of AD development.

It has also been shown that in the space of the synaptic cleft, the migration of Ca⁺² and Mg⁺² and Zn⁺² ions is inhibited or antagonized by bivalent toxic chelating ions such as Hg—Cd—Sn (bivalent), thallium, or trivalent Al³⁺, As⁺³ or hexavalent (Cr⁺⁶). Copper Cu²⁺ is a powerful competitor of Zn⁺² in the synaptic cleft and can cause rupture of the Zn/Cu equilibrium in whole brain tissues, liver and pancreas tissues, and will affect the neuronal, liver or pancreatic activity, and will increase the Aβ formation in the context of Alzheimer's Disease, inducing anisotropy in the molecular BBB integrity.

The most common genetic risk factor for Alzheimer's Disease is the e4 allele of the gene for apolipoprotein E (APOEε4), which is present in approximately 50% of individuals with AD. APOEε4 heterozygosity triples the risk of AD compared with non-carriers; homozygotes have a sevenfold risk. Other less prevalent risk genes and familial tendencies have also been identified.

Mutations in the genes for amyloid precursor protein (APP, on chromosome 21), presenilin 1 (PS1, chromosome 14), and presenilin 2 (PS2, chromosome 1) cause autosomal dominant early-onset AD. These mutations account for the majority of familial midlife-onset AD, but represent less than 5% of all AD cases. Sortilin 1 (SorL1) mutations cause late-onset AD.

AD's core neuropathologic findings include extracellular amyloid plaques, intracellular NFTs, synaptic deterioration, and neuronal death granulovacuolar degeneration in the hippocampus and amyloid deposition in blood vessels. Cerebral Amyloid Angiopathy (CAA) may also be seen on tissue examination under nMRI, but are not required for the diagnosis. The “amyloid cascade” hypothesis posits that amyloid plaques interfere with synaptic activity and initiate a series of downstream effects that cause increasing inter- and intraneuronal dysfunction and, ultimately, cell death.

Although amyloid plaques may be sub-classified according to their composition, all contain forms of β-amyloid protein (Aβ). Aβ is an amino acid peptide formed by proteolytic cleavage of APP by β- and γ-secretase. The main products of this cleavage are Aβ₁₋₄₀ and Aβ₁₋₄₂. A relative surplus of Aβ₁₋₄₂ predisposes toward amyloid aggregation into oligomers and fibrils, which assemble into amyloid plaques.

An important role for amyloid in Alzheimer's Disease pathophysiology is implied by the fact that the proteins encoded by APP, PS1, PS2, SorL1, and APOEε4 are all associated with amyloid generation, processing, and/or trafficking. However, several lines of evidence indicate that amyloid plaques are not the primary cause of AD. Amyloid plaque burden (a) can be found in cognitively normal adults, (b) does not correlate with degree of cognitive impairment in individuals with AD dementia and, (c) is associated with cognitive improvement in some AD mouse models.

Tau, a protein involved in microtubule assembly, is essential for normal axonal growth and neuronal development. However, hyperphosphorylated tau protein aggregates into helical filamentous NFT that are deposited preferentially within neurons of the mesial temporal lobe (especially hippocampus), lateral parietotemporal region, and the frontal association cortices. The critical role of NFT in AD pathophysiology is suggested by the correlation between location and density of tau NFT and the symptoms and severity of AD dementia moreover, some studies have demonstrated that Aβ oligomers are not toxic unless tau proteins are also present.

The distribution of neuronal cell death and synapse loss is similar to that of NFT. In typical AD, the death of neurons in the nucleus basalis of Meynert leads to a deficit in acetylcholine (Ach), a neurotransmitter involved in memory. This cholinergic deficit is the target of most current treatments. In the brainstem, loss of median raphe and locus coeruleus neurons leads to deficits in serotonin and norepinephrine, respectively. Abnormal cerebral serotonergic and adrenergic activity likely contribute to dysphoria and insomnia in AD

In addition, certain toxicological considerations indicate that the pathology of Alzheimer could propagate to other neuronal tissues in a transmissible prion mode increased by the ROS oxidative stress, by endogenic contaminants (with cholesterol LDL as “monomer” promoter prion of Aß plaques) and exogenic molecules.

Aβ-APOEε4 plaques are thus mainly, at different molecular stages, resulting from cholesterol molecular pathology initiated in membranes, wherein the status of protons as donors or acceptors is strongly perturbed by the process of CHO esterification, compromising the cleft integrity.

Several references, lastly in March 2019, indicate that laboratories leading in research into pharmaceutical pathways aimed at reducing or destroying amyloid plaques and Tau proteins have abandoned their efforts through failure. Thus, despite considerable efforts by laboratories, known medications still only halt the symptomatology of Alzheimer's disease temporarily, but have side effects from low to severe stage.

The present approach aims to introduce novel pharmacological engineering, as compositions, protocols and experimental physics processes, for mitigating and treating such pathologies. ¹https://www.sciencema.org/news/2019/03/another-major-drug-candidate-targeting-brain-plaques-alzheimer-s-disease-has-failed

### SUMMARY OF INVENTION

According to an aspect of the invention, there is provided a composition comprising zinc Zn-FLNT4 and silicon SiHR57 in the form (3(CH₃Si(OH)₃—R(COO X)) for use as a medicament or in therapy. More particularly, according to an aspect of the invention, there is provided a composition comprising zinc Zn-FLNT4 in the form of an organic compound soluble in water and having a carbonyl function complexed with zinc, and silicon SiHR57 in the form of (3(CH₃Si(OH)₃—R(COO X)), wherein x is selected from the group comprising sodium, potassium and lithium, for use as a medicament or in therapy.

“zinc Zn-FLNT4” described herein will be understood as an organic compound soluble in water and having a carbonyl function complexed with zinc. The structure and process for the preparation of organometallic complexes including zinc Zn-FLNT4 is described in detail in France Publication No. FR2708002 and PCT Application Publication No. WO/1996/023089, each of which are hereby incorporated by reference in their entirety.

It will be understood that the silicon atom in (3(CH₃Si(OH)₃—R(COO X)) is hypervalent (pentavalent). This principle is well established in the field of chemistry. For example, https://pubs.acs.org/doi/10.1021/ja0162616; http://www.f.u-tokyo.ac.jp/˜kanai/seminar/pdf/Lit_T_Ito_B4.pdf; and https://www.scripps.edu/baran/images/grpmtgpdf/Hafensteiner_Nov_03.pdf, each of which are hereby incorporated by reference in their entirety.

In a variant of these embodiments, the zinc Zn-FLNT4 component is preferably associated with a D-fructose molecule.

In embodiments, the organic compound soluble in water and having a carbonyl function may be at least one selected from a sugar, an oligosaccharide, a disaccharide, an enzyme, a coenzyme, a protein, a peptide, an amino acid, a hormone, a steroid, a prostaglandin, a neurotransmitter, a vitamin, an ester, a carboxylic acid, a ketone, an anhydride, and an antibiotic.

The composition may be for use as a medicament or in therapy, in combination with a whole lung lavage procedure.

Embodiments of the composition may be for use in a method for the treatment of neurodegenerative and cardiovascular diseases. The method may use one or more further compositions, for instance a second composition comprising cyclodextrins (αβγ) and/or a third composition comprising ursodeoxycholic acid, and may use one or more further resources, for instance master stem cells.

Alternatively, in at least some embodiments, the composition may be for use in a method for mitigating neurodegeneration associated with exposure to at least one selected from solar energetic particles, galactic cosmic rays and extra-terrestrial radiation. These embodiments of the composition may usefully be incorporated into one or more human food supplement(s) for delivery.

According to another aspect of the invention, there is also provided a method of aligning the spin of molecules in embodiments of the composition presented hereinabove and described hereinafter, comprising the steps of locating at least one conduit adjacent a source of electromagnetic radio waves (EM-RF), causing the composition to flow through the conduit, and generating an EM-RF with the source, for subjecting composition in the conduit to the generated EM-RF. The conduit may for instance be the inlet and outlet of a controlled-rate syringe.

In an embodiment of the method suitable for pathology mitigation, for minor or early pathologies, and for longer-term treatment associated with high patient susceptibility to EM-RF, the EM-RF may be generated with intensity in the range 0.05 Tesla to 0.3 Tesla. Alternatively, in an embodiment of the method suitable for advanced stages of pathologies, the EM-RF may be generated with intensity in the range 1.5 Tesla to 3 Tesla.

Embodiments of the method consider the generation of the EM-RF under a variety of forms, each dependent upon one or more of patient morphology and cellular aspects, pathology and stage thereof, availability of equipment and more, with such forms including a (B) permanent EM-RF, a pulse-modulated EM-RF or an oscillating EM-RF.

An embodiment of the method may comprise the further steps of administering the EM-RF-subjected composition to the patient, locating at least an area of the patient including the administered composition, typically the patient's cranium or a portion thereof, adjacent a magnetic resonance imaging (MRI) apparatus, and generating a second EM-RF with the MRI apparatus, for subjecting the area including the administered composition to the generated second EM-RF. In a variant of this embodiment, the second EM-RF may be generated with intensity in the range 1.5 Tesla to 3 Tesla.

Embodiments of the method consider the administration of compositions as described herein through one selected from oral ingestion, endonasal or intranasal spray, dermal probe, transdermal patch and intraperitoneal or intracranial injection.

According to a further aspect of the invention, there is also provided a cyclodextrin particle for delivering the composition presented hereinabove and described hereinafter.

Embodiments of the cyclodextrin particle may be shaped as a three-dimensional cage with at least one aperture, and may optionally be shaped as a torus.

Embodiments of the cyclodextrin particle may further comprise silicon-germanium nanoparticles or picoparticles located on an inner surface of the cyclodextrin particle.

Embodiments of the cyclodextrin particle may further comprise Zinc carbonyl located on an outer surface of the cyclodextrin particle.

Other aspects of the present invention are as stated in the appended claims.

### BRIEF DESCRIPTION OF DRAWINGS

Embodiments of the present invention will now be described by way of example only, with reference to accompanying drawings, which are:

FIG. 1 provides chemical and three dimensional representations of cholesterol (CHO);

FIG. 2 shows electronic imaging of healthy tissues on the right and tissues affected by prions on the left;

FIG. 3 provides a three dimensional representation of pathological protein fragments;

FIG. 4 illustrates prions and fragments in a diseased human context [Mm 386.65 386 kDa 386650 Da];

FIG. 5 illustrates the concept of self assembly of cholesterol monomers;

FIG. 6 provides a chemical representation of ursodeoxycholic acid [Mm 395.56 395560 Da 395 kDa];

FIG. 7 illustrates the concept of spin alignments under the application of magnetic field and radio frequency, from an anisotropic distribution on the left to isotropic spin alignments on the right;

FIG. 8 provides a three dimensional representation of human haemoglobin structure, with a and R subunits respectively in red and blue, and iron-containing heme groups in green;

FIG. 9 illustrates the formula and shape of haemoglobin;

FIG. 10 provides a chemical representation of active heme groups Fe⁺²;

FIG. 11 illustrates a torus of cyclodextrins embodied as three-dimensional cage molecules, adapted to a magneto hydro dynamics (MHD) process field;

FIG. 12 illustrates cyclodextrin polymers adapted to magnetic vectors;

FIG. 13 is a logical diagram of a plasma toroidal apparatus;

FIG. 14 is a logical diagram of a nano- and pico-particle production process; and

FIG. 15 is a logical diagram of a MHD generator motor process.

### DETAILED DESCRIPTION OF DRAWINGS

The inventor has been working for many years on the biophysical aspects of biological material and therefore DNA-RNA (intronic quantum biology). The intronic quantum concept of experimental theory of bio-physics mechanisms associated with DNA-RNA sequences in living genetic material is considered a prominent problem.

It has been shown that human DNA encodes only 5% of the classical biological reactions known as exonics. 95% of the germplasm appears to be missing visible participation in the biological material protein. This non-coding part of our DNA (95%) has been defined as intronic RNA. An intron is a non-coding DNA sequence interrupting the coding sequence of a poly partite gene (opposed to exon). An intron can be seen as a fragment of a gene located between two exons. Introns are present in immature mRNA and absent in mature mRNA. For the past 10 years, biologists have been confident that intronic non-coding parts play an important role in genetic and protein synthesis reactions, contrary to accepted initial ideas that the intron part of DNA serves no purpose.

These immature RNA introns could induce real genetic reactions, intervene in immune defense reactions, and disappear once their mission is complete. One might consider immature RNA introns that can be assimilated to particular biological messengers. Immature RNA introns could transmit their information according to mechanisms as well as biophysical (quantum signals), non-classical chemical and therefore molecular principles. Immature DNA-RNA sequences may be considered to encode real, but stealth, proteins which dematerialize in the medium biological medium, upon receipt of the signals transmitted by the immature RNA sequence by the genetic material.

On this basis, several researchers have envisaged a non-molecular, but bio-physical system, in which the immature RNA genetic material transmits quantum-type signals at specific frequencies, mainly in the ultraviolet (UV) spectrum. One of the strong ideas of the experimental theory provides for the presence of an N—H (plasma of hydrogen) within the DNA double helix, which has been developed in a third catenary or strand of DNA. This plasma alone could explain the emitted or associated UV radiation of the mRNA material, which would bio-quantitatively encode stealth proteins. Describing these stealth proteins may be 10⁻³ s to 10⁻⁵ s does not prove that they do not exist, but have the kinetic time to transmit quantified information flows. The logical consequence of the amplification of these quanta of energy lies in the collateral reasoning for the maintenance of the emitted signal, in order to avoid entropy of the biological material.

There are very few elements in our biological material that could sustain such signals. The inventor has privileged natural elements such as Silicon and Germanium, or Sulfur S32, therefore tetravalent elements, wherein both Silicon and Germanium possess opto-quantum properties, i.e. they accept radiation from a source or emit it under precise experimental conditions.

The effects of silicon on different organs and functions are scarcely known, although there is growing evidence of its role in mineralization, cardiovascular system, skin, immunity, as well as brain (Jurkic et al., 2013). It has been suggested that silicon consumption in western countries is reduced (Robberecht et al., 2009). Thus, the addition of this mineral to certain food, in addition to improve nutrition will contribute to improve different health key functions.

Studies carried out with organic Silicon G57 by our research have demonstrated the great antioxidant and neuroprotective capacity of this metal (Garcimartin et al., 2014; 2015a). In addition, the consumption of organic Silicon G57 improved the brain antioxidant status of rats intoxicated with aluminum, decreased learning capacities of these animals (Gonzalez-Muñoz et al., 2017). We have also observed the effect of this product on hyperlipidemia, as well as a reduction in the fatty liver progression in animal model when silicon is added to meat matrices as a functional ingredient (Garcimartin et al., 2015b; 2017).

The present approach thus considers compositions based upon Zinc FLNT4, and Silicon SiHR57 (derived from Silicon G57), in simultaneous activities on several processes involved in the development of the AD and stages, as a multi-target adapted to inhibit the AChE enzymes, β-secretase and γ-secretase activities; and to reduce the fibrillogenesis of the βA peptide, in order to attenuate the intracellular generation of free radicals and the release of nitric oxide (NO) by the microglia.

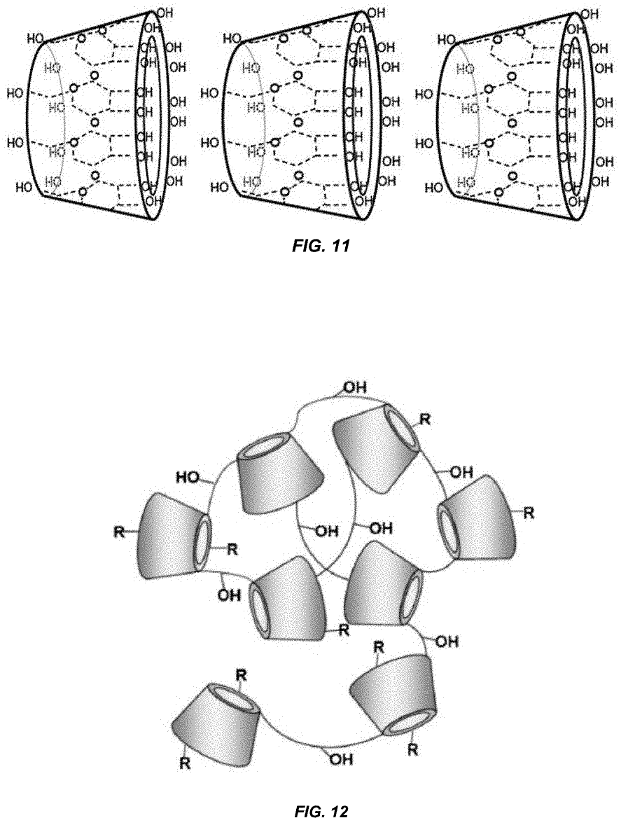

The present approach considers the following compositions:
SILICON HR 57(SiHR57)-01, composed of ZnFLT4 (cyclic) formula Zn associated with D-Fructose molecule on C═O Carbonyl radical, associated with a silicon compound (3(CH₃Si(OH)₃—R(COO X)), wherein X can be sodium (Na), potassium (K) or Lithium (Li);
SILICON HR 57(SiHR57)-02, with the same composition as SiHR57-01, associated with cyclodextrins (αβγ) as illustrated in FIG. 11 and shown in polymeric form n(20-200) in FIG. 12;
SILICON HR 57(SiHR57)-03, with the same composition as SiHR57-01, associated with ursodeoxycholic acid; and
SILICON HR 57(SiHR57)-04, with the same composition as SiHR57-01, associated with both cyclodextrins (αβγ) and ursodeoxycholic acid.

Zn-FLNT4 (cyclic) Zn Carbonyl is here considered a solvent of Aß plaques and ROS residues. The ZnFLNT4 (cyclic) compound may contain Lanthanides Ln³⁺ (or Gd³⁺ or others), with LN in the expression ZnFLNT4 associated with Lanthanides, and T4 because the product has been used in cancerology (Grandi-Assoun et al, WO/1996/023089). The respective molecular weights of cholesterol and ursodeoxycholic acid are very close, aromatic cycles and three dimensional development are also similar, but ursodeoxycholic acid wears 3OH-hydrophilic radicals, contrarily to cholesterol with only one OH radical.

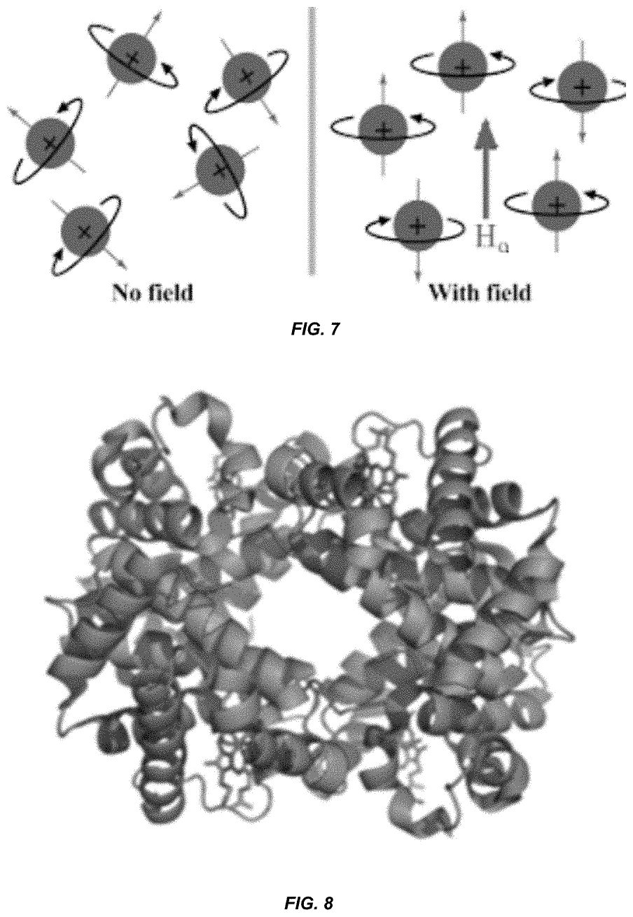

The present approach considers, by way of protocols, subjecting both the SiHR57 composition and the patient to a variety of magnetic fields, optionally in combination with pluripotent (master) stem cells for regenerative purposes. A first ‘Phase I’ protocol relies upon the use of magnetic fields (B permanent Nd alloys, 0.03 Tesla to 0.1 Tesla, up to 1.5 Tesla to 3 Tesla) or pulsed-modulation or oscillating mode) as external means of collateral and synergetic treatment coupled to the SiHR57 composition, in order to engage preliminary spin alignments, the principle of which is illustrated in FIG. 7, under the application of low magnetic field and RF (phase of coherence of spins). One or more electro-magnetic (EM) condensate RF radio frequencies may be selected from the following ranges calculated with the algorithm of Geesink and Meijer (NeuroQuantology, March 2016, Issue 1, pp. 106-125):
4-8 Hz: 4.0, 4.22, 4.5, 4.74, 5.06, 5.33, 5.70, 6.0, 6.32, 6.75, 7.11, 7.59 Hz
32-61 Hz: 32.0, 33.7, 36.0, 37.9, 40.5, 42.7, 45.6, 48.0, 50.6, 54.0, 56.9, 60.75 Hz
64-122 Hz: 64, 67.5, 72, 75.78, 81, 85.3, 91.18, 96, 101.1, 108.0, 113.8, 121.5 Hz
255-487 Hz: 256, 269.8, 288, 303.1, 324, 341.2, 364.7, 384, 404.5, 432, 455.1, 486 Hz
16.3-31.2 kHz: 16.38, 17.25, 18.43, 19.40, 20.74, 21.84, 23.34, 24.58, 25.91, 27.65, 29.13, 31.10 KHz
16.7-32 MHz: 16.77, 17.66, 18.87, 19.86, 21.24, 22.36, 23.90, 25.17, 26.53, 28.31, 29.83, 31.85 Mhz
4.2-8.2 GHz: 4.293, 4.520, 4.831, 5.085, 5.437, 5.724, 6.119, 6.443, 6.792, 7247, 7.636, 8.154 GHz.
1.1-1070 THz: 1.10, 1.158, 1.237, 1.302, 1.3391, 1466, 1.566, 1.649, 1.738, 1.855, 1.955, 2.088 Thz 2.20, 2.316, 2.474, 2.604, 2.783, 2.931, 3.133, 3.298, 3.475, 3.710, 3.909, 4.175 THz 4.40, 4.633, 4.948, 5.208, 5.566, 5.863, 6.266, 6.597, 6.950, 7.420, 7.819, 8.350 Thz 8.80, 9.266, 9.897, 10.42, 11.13, 11.73, 12.53, 13.19, 13.90, 14.84, 15.64, 16.70 THz 17.59, 18.53, 19.79, 20.83, 22.26, 23.45, 25.06, 26.39, 27, 80, 29.68, 31.28, 33.40 THz 35.19, 37.06, 39.59, 41.66, 44.53, 46.90, 50.13, 52.78, 55.60, 59.36, 62.55, 66.80 THz 70.38, 74.13, 79.18, 83.33, 89.05, 93.80, 100.3, 105.6, 111.2, 118.7, 125.1, 133.6 THz 140.8, 148.3, 158.4, 166.7, 178.1, 187.6, 200.5, 211.1, 222.4, 237.5, 250.2, 267.2 THz 281.5, 296.5, 316.7, 333.3, 356.2, 375.2, 401.0, 422.2, 444.8, 474.9, 500.4, 534.4 THz 562.9, 592.9, 632.7, 666.5, 712.4, 750.4, 802.0, 844.4, 889.6, 949.8, 1000.8, 1068.8 THz

A comparison of frequencies extracted from biological studies (x), with frequencies calculated via the Geesink and Meijer mathematical algorithm (y), and their respective percentage differences (z), are presented hereafter in the format (x, y z):

Positive effects:

| Hz: | MHz: | kHz: | GHz; |
|---|---|---|---|
| 100.0; 101.1; − 1.09% | 0.500; 0.498; + 0.40% | 33.00; 32.75; + 0.73% | 46.00; 45.79; + 0.46% |
| 110.0; 108.0; + 1.85% | 1.000; 0.995; + 0.50% | 35.00; 34.50; + 1.45% | 51.05; 51.54; − 0.95% |
| 120.0; 121.5; − 1.24% | 1.500; 1.490; + 0.67% | 448.0; 442.4; + 1.27% | 60.13; 61.09; − 1.57% |
| 150.0; 151.6; − 1.06% | 3.000; 2.990; + 0.33% | | 61.20; 61.09; + 0.18% |
| 160.0; 162.0; − 1.24% | 3.770; 3.730; + 1.07% | | 64.50; 65.23; − 1.12% |
| 200.0; 202.2; − 1.09% | 5.000; 4.970; 0.60% | | 65.00; 65.23; − 0.35% |
| 300.0; 303.1; − 1.02% | 8.000; 7.960; 0.50% | | |
| | 20.00; 19.90; 0.50% | | |
| | 21.20; 21.24; 0.19% | | |
| THz: | | | |
| 0.129; 0.1305; − 1.15 | 452; 449.8; + 0.49% | 627; 631.3; − 0.68% | |
| 2.300; 2.316; − 0.69% | 456; 449.8; + 1.38% | 629; 631.3; − 0.36% | |
| 3.680; 3.710; − 0.81% | 470; 473.8; − 0.80% | 630; 631.3; − 0.21% | |
| 390; 399.5; − 2.38% | 515; 505.6; + 1.86% | 632.8; 631.3; + 0.24% | |
| 393; 399.5; − 1.63% | 503.96; 505.6; − 0.32% | 638; 631.3; + 1.06% | |
| 395; 399.5; − 1.13% | 505.86; 505.6; + 0.05% | 670; 674.0; + 0.59% | |
| 404; 399.5; + 1.13% | 530; 532.5; − 0.47% | 675; 674.0; + 0.15% | |
| 415; 420.8; − 1.38% | 532; 532.5; − 0.09% | 700; 710.1; − 1.42% | |
| 420; 420.8; − 0.19% | 560; 561.0; − 0.18% | 740-750; 747.6; − 0.35% | |
| 445; 449.8; − 1.07% | 590; 599.1; − 1.52% | | |
| 450; 449.8; + 0.04% | 605-610; 599.1; + 1.40% | | |

Negative effects:

| Hz: | MHz: | Ghz: |
|---|---|---|
| 6.600; 6.540; + 0.92% | 835.0; 827.2; + 0.94% | 2.450; 2.479; − 1.17% |
| | | 9.417; 9.351; + 0.71% |

A first ‘Phase II’ protocol relies upon the use of magnetic resonance imaging (MRI) apparatus to induce spin alignments, as a preliminary method to facilitate the transportation and dissolution of prion species out of the BBB and brain tissues, by means of solvents and captors of problematic molecules ROS reversing the effect of toxic agents. Use of external rotating and oscillating magnetic fields B (extracranial) permanent (lanthanides alloys) or pulsed(solenoids). The present approach further considers the use of a vertical or longitudinal cylinder (entire body introduction), for instance composed of several magnetic layers (possibly 7 to 10) generating permanent or pulsed vector fields, with each part of a cylinder isolated from others with bismuth foil or plaques.

The present approach may be taken as a reverse cholesterol LDL methodology for mitigating the toxic formation of Aβ plaques-tangles-APOεE4 and prevention of Cardio Vascular Diseases CVD and CVA(strokes) but also the transmission of prion molecules in total neural brain tissues-CNS, Aβ plaques (as responsible of future molecular diseases and inflammation and immunodeficiency).

The mechanisms of ROS (Oxidative Stress) can be mitigated if patients can follow the protocols with 2 to 3 intensive sessions per month or more, and prevented through periodical sessions justified by genetic factors. Under the Phase I protocol, a patient may wear a monitored and programmed multi-geometric device around their head, potentially daily and for up to several hours, in order to target the CNS neural brain areas to be treated. Real-time MRI imagery under the Phase II protocol rapidly establishes the best vectors and routes of the SiHR57 composition, in order to decrease the stages of Alzheimer Disease.

The methodology uses the dissolution properties of several compounds mainly ZnFLNT4 (cyclic molecule) associated to Zinc, as main solvent of Aβ plaques and prion fragments, Tau proteins (self-assembly or polymerized structures and degraded form) APOEε[4], hemosiderin degraded, CHO polymerized form, self-assembly.

Silicon complex compound as (protector), cyclodextrins(αβγ) as secondary (solvents), cyclodextrins as monomer and/or polymerized form and/or ursodeoxycholic acid as solvent (FIG. 6 provides a chemical representation of ursodeoxycholic acid), products active in the dissolution of cholesterol LDL self-assembly and polymerized form and heavy proteins and Ap amyloid or assimilated plaques or on prion molecules.

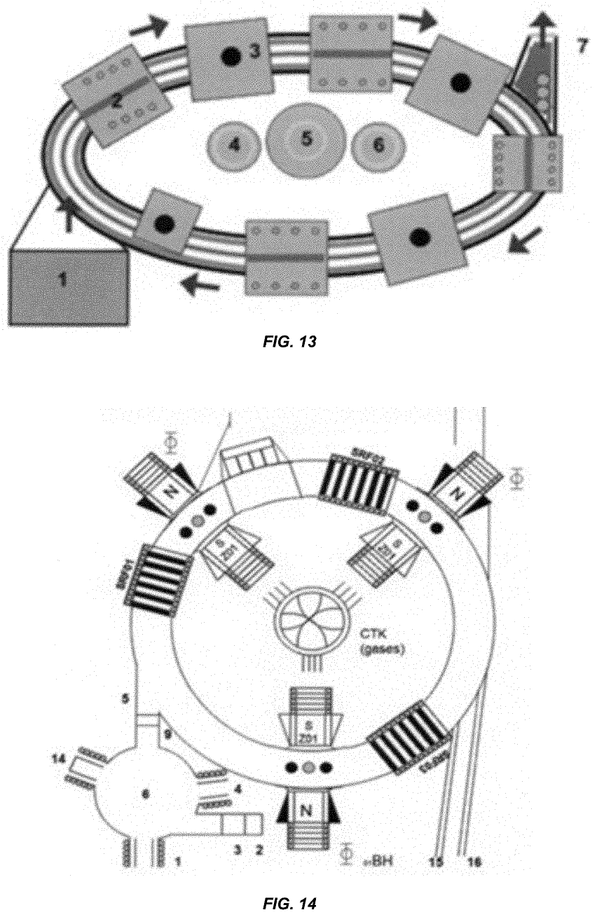

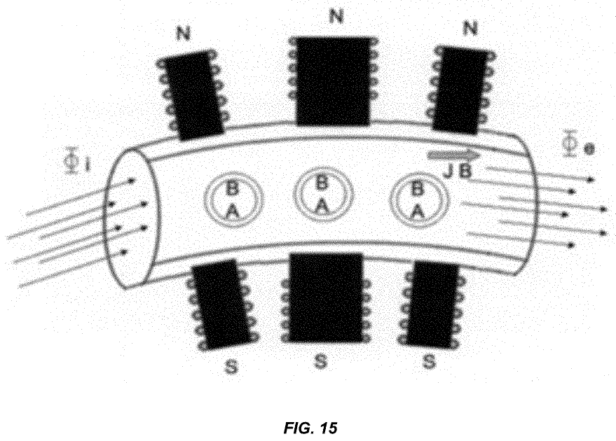

Before reconstruction of neuronal tissue with the use of pluripotent stem cells applied (outside BBB) to the neuronal tissue and mainly to glial cells astrocytes, under magnetic NMR and electro-Magnetic frequencies (radio waves) and induced by MHD-MGD (FIGS. 13, 14, 15) kinetics and permanent magnetic field B (alloys of Lanthanides) Ni—Co—Fe, others or pulsed (diodes) or oscillating mode (solenoids) under external transcranial 3D apparatus MRI instruments.

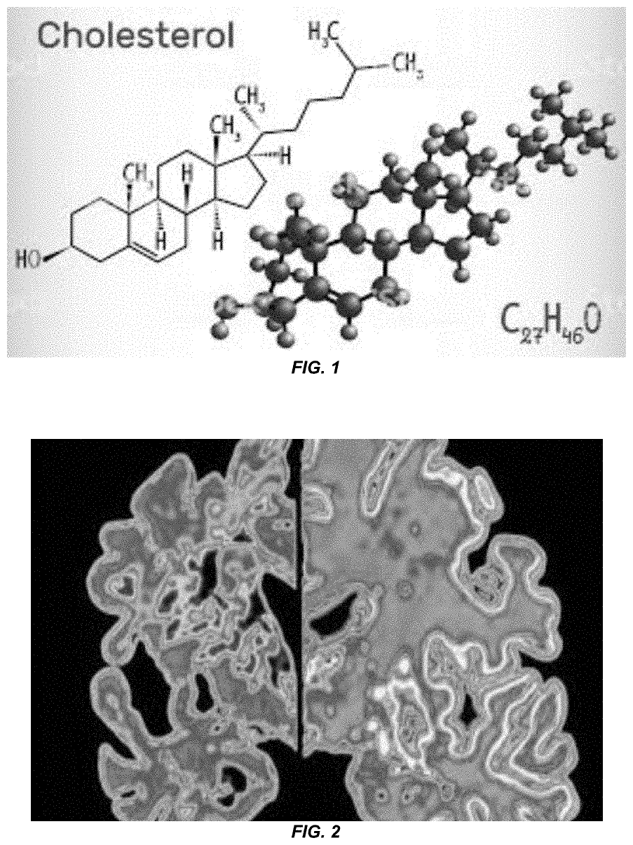

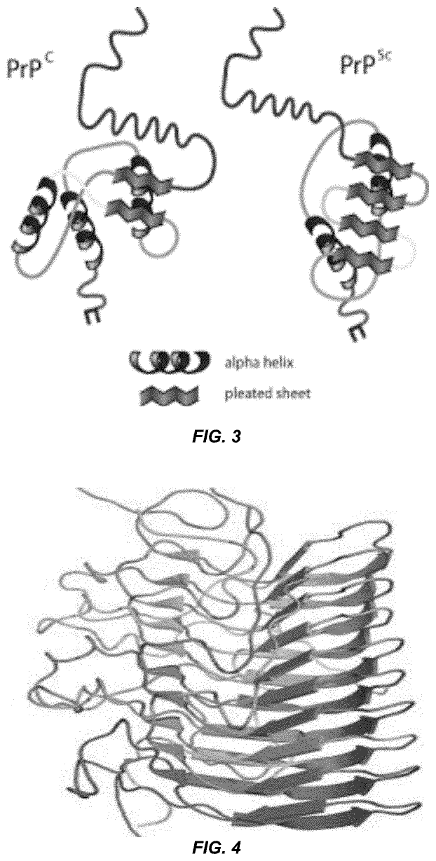

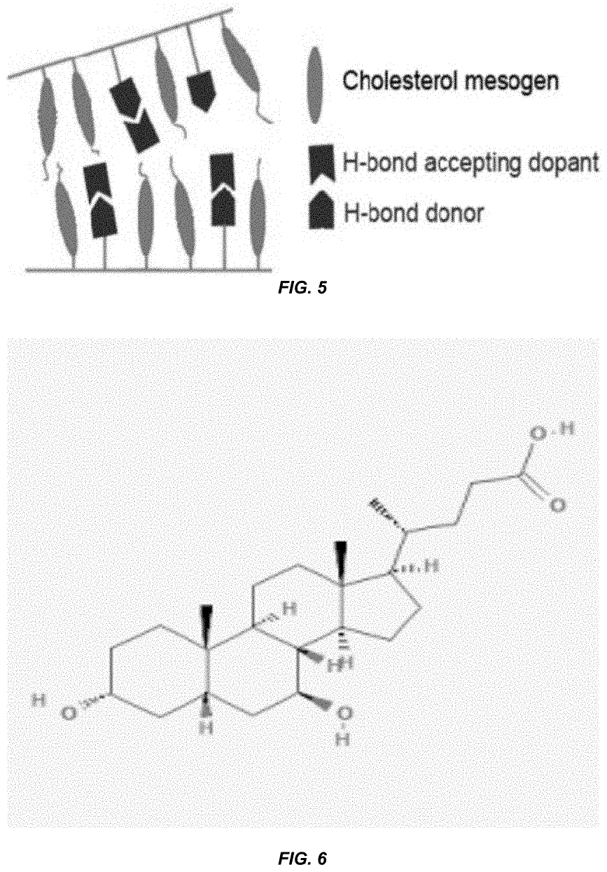

A rebuilding protocol can be considered with master stem cells, but it should be executed only after the dissolution process of Aβ plaques and prion fragments, Tau proteins (self-assembly or polymerized structures and degraded form) FIGS. 1, 2 and 3, APOEε[4], hemosiderin degraded (NMR imaging control).

Cyclodextrins and Haemoglobin (Hb); Hemosiderin (in contaminated tissues) are active under, and can be eliminated from BBB though, magneto hydro dynamics (‘MHD’)—magneto-gas-dynamics (‘MGD’) processes: protons transportation H⁺ concept and process as molecules in three dimensional cages (FIGS. 11 and 12) with para- and diamagnetic susceptibilities, but also ferromagnetic properties (Hb heme nuclei).

The BBB is considered as “a physical tube or geometrically developed sphere (if we consider all the brain as final structure) interface membrane or an envelope”, in the process of neurulation, one can verify that the primitive laminar (neural tube), will be transformed under genetic tensors, in ectoderm, endoderm and mesoderm, with final shape of a tube.

BBB-CNS is assimilated to a MHD system where heme ions Fe⁺² (atomic ions current ni) are circulating and developing magnetic fields (vectors). When the ROS is increasing the Fe³⁺ will create sub-aggregates through the BBB and the migration of heme Fe³⁺ under ROS in the neuronal cleft will be considered as the collateral source of biodegraded molecules (in addition to Aβ plaques and prion fragments) blocking the cleft communication and neurotransmitters mediators.

Haemoglobin presence in the cortex under active ROS will be degraded and will generate collateral products in neuronal tissues, prions, tangles and contaminants fibrinogens), whereby the BBB will considerably disturbed, the synaptic activity will be perturbed or destroyed. The degradation of Haemoglobin in brain tissues results in the deposition of hemosiderin, insoluble form of Fe⁺² which is a major form of iron-storage protein and closely related to neurological disorders—and strokes—the pumps membrane are blocked, the mechanical disorders has been verified by micro pumps at lab (time species transport femtosecond) by several authors. The elaboration of a preventive or curative drug (or even a functional food) must by its components be able to block ROS contaminants such as Aluminium Al³⁺ and other toxic metals (including Mercury, Hg²⁺ and especially in the form of (Mono Methyl Mercury) and others contaminants as well as Dioxins.

The SiHR57 compositions can fight against oxidative stress but also potentially dissolve the Aβ protein plaques, but also block the adverse effects of free cholesterol as promoter or in its aggregated form self-assembly which will become cofactor of the presence of Aβ protein.

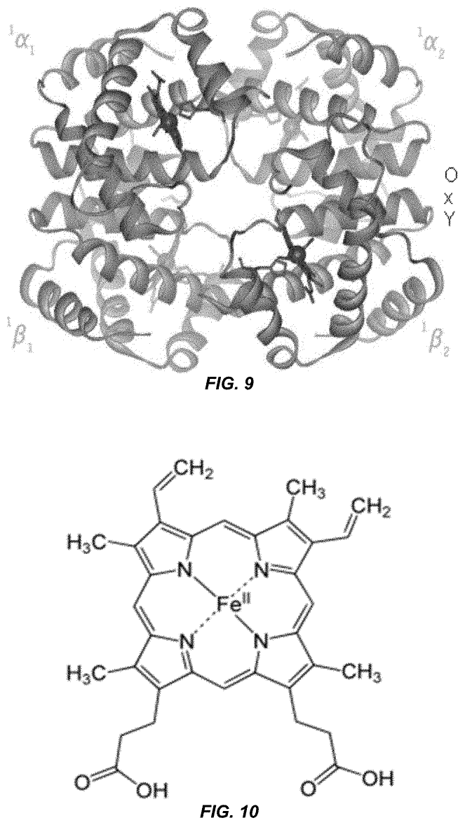

The presence of Haemoblogin (heme nuclei) as illustrated in FIGS. 8, 9 and 10 (n)Fe²⁺ in the synaptic cleft as well as in the exchange mechanisms with BBB, and in astrocytes environment, and bind to BBB, as well as the production of super oxidant H₂O₂ gives rise to peroxidised compounds (ROO—) in FeO—Fe₂O₃ plus O₂ native and hemosiderin paramagnetic iron form (pathological molecule) in the presence of the super oxidant H₂O₂. Others will produce Fe⁺² ion and the creation of molecular native oxygen O₂ (macroscopic experiment done (in chemical reactor) which indicates in a semi-empirical way that the biodegradation products of the Haemoglobin are high molecular weight buffers that are major barriers to exchange in BBB membranes and synaptic clefts that may be considered as “membrane like”.

Regarding hemosiderin the degraded protein with iron content can be verified with XRF (radiation tools) Argonne Laboratory DOE USA—hemosiderin is iron paramagnetic molecule who might be used in MHD-magnetic vectors and be eliminated when solved through BBB.

Our approach is to create SiHR57 products (several formulations and protocols) that will aim at preserving a reducing state in membranes or assimilated to membranes by means of molecules and atoms that act as reducing agents and particularly Zinc-Zn⁺² and Silicon Si⁺⁴Our approach consists in activating the mechanisms of dissolution of the long protein chains Aß and fibrinogens and thus reducing them to structures less sensitive to the states of peroxidation, the presence of agents Zn⁺², Si⁺⁴ reducers can chemically allow it, so the medium will be protonated H₃O⁺ non peroxidised. [—O—O—] thus reducing oxidative stress ROS.

In the pre-treatment of prions and plaques and fibrinogen products one will first apply permanent magnetic B (Ln³⁺ alloys and solenoids(pulsed) and EM (pulsed) vectors on preventive or curative or fibrinogen monomers in a quasi-polymerized form.

It should be noted that the 1.5-3 T fields will cause spin alignments of biomolecular species in fibrinogen form, such as Aß plaques or aggregated cholesterol complexes.

This phenomenon is possible at the atomic level by aligning the spins. In the phenomena of polymerizations of the plaques and/or prions there exists a spin-orbit coupling which preserves the anisotropy of unstable and/or peroxidized molecular forms.

Spin orbit coupling is a quantum-magnetic dipole-type discussion in a magnetic field. Interesting experience with Ln⁺³ (lanthanides) including Gd³⁺ and labeled Tc⁺³. Exposures to 1.5 T-1.5 T and well-calibrated exposure time limits, in order to avoid possible overheating of water and tissues. An important consideration is to limit the background noise of the molecule of water as much as possible, compared to the signal.

The protocols can be integrated into an exogenous or endogenous therapeutic approach (nanoprobes) to be coupled with simple or complex chemical and molecular agents orally, intravenously, intracranial or intranasal or by endothelial nasal application via pulsed aerosols. Pre-treatment in the laboratory of compositions by permanent magnetic field B-Lanthanides alloys Permanent or solenoids/Pulse −0.05 T-0.1 T-1.5 T-3 T Magnetic vectors and/or EM and/or 1.5 T-3 T. The pre-treatment excites the solutions to be administered and allows for testing the stability of molecular conformations. The homogeneous state of the solutions elaborated is ensured by ultrasonic apparatus.

Analyzes (elucidation of NMR structures-GC-MS-LS) make it possible to gauge the couplings of active products in order to obtain a better response in the cerebral environment. In order to optimize the active molecules SiHR57, the goal is to use in the very short term (a few weeks) non-invasive technologies that safely allow to follow the real-time course of drugs administered in animals and/or the human (human neuro imaging) and therefore to better target and know with extreme precision the active areas and the response of the neuronal environment in the presence of drugs developed by the laboratory. It is possible to accurately determine the ability of products to cross BBB and gauge the pharmacokinetics of products and determine with great accuracy the allowable quantity of drugs and thus the bioavailability of drugs destinated to brain affected areas. It is also possible to determine the intrinsic activity of drugs and control the results obtained by imaging. Neuro-imaging and cardiovascular imaging will improve the drugs produced by the laboratory and increase the treatment potential for patients with neurodegenerative diseases.

If insoluble forms Fe³⁺ ions (Fe₂O₃) is generated by the permanent ROS, Fe₂O₃ are not active ions structures submitted to MHD only free ions, in the case of BBB process (in-out[elimination]). One makes the difference between IONIC forms of atoms A⁺ⁿ (room temperature) and ionized states A, I, II, III experiment temperature associated to radiative emissions and transfers. Associated to Saha-Boltzmann equations of Thermal Equilibriums LTE-NLTE-PLTE temperatures 900 K to 30′000 K and more IV. Ions (H⁺, (D⁺[H—H]⁺), Fe²⁺, Ca⁺², Zn²⁺, Mg²⁺, Ln³⁺ . . . ) can be elected to MHD-MGD processes, as atomic species seeded in the plasma state chemistry-physics consideration (LTE-PLTE-NLTE).

Cyclodextrins-polymerized forms, Hb, ZnFLNT4, (other cyclic molecules (aromatics), can be elected to MHD-MGD., synthetic cyclic, nanoparticles NP and PP picoparticles-(destinated to Neuro Space Research).

The association of the four compounds inside the product SiHR57-01-02-03 (protocols) (NanoP-PicoP) particles (molecules) or atoms are used in order to through or diffused in the BBB and clean the barrier and reach neuronal tissues organization and dissolve the Aβ plaques and ROS residues (different routes are proposed for the drug route, included N2B).

The active molecules of the compositions are elucidated with NMR-GC-MS-LC-MS spectroscopy tools.

The first mission of clean-up of the synaptic cleft interface mediators and dissolution and degradation of Aß amyloid plaques, τ tau proteins (self-assembly or polymerized structures and degraded form), prions, tangles and PrP fragments, hemosiderin degraded, copper oxide, and APOEε[4] species in the cortex and hippocampus areas, others, re-activation of the connection area reconstruction of the synaptic network, and neurons nodes and poly-matrix organizations, transformation of astrocytes pool in mature neurons, reconstruction by the mean of pluripotent (master) stem cells and under magnetic B permanent or pulsed and electro-magnetic vectors, as well as Magneto-Hydro Dynamics MHD (FIGS. 13, 14, 15) inside and outside BBB and in general blood circulation and associated at pharmacological vectors applications (SiHR57-SILICON HR 57). Solvents are Zn-FLNT4cyclic(D-fructose), others cyclic compounds, cyclodextrins (αβγ) cyclic(toroids-torus) and/or polymerized configuration of Cyclodextrins nij[2-400] and ursodeoxycholic acid.

Elucidation and evaluation of the possible MGD (Magneto-Gas-Dynamics) applied on Hydrogen atom and bonds to Nitrogen- and in nucleic bases RNA-DNA. Hydrogen bonds at (steady state kinetics) LTE (Local Thermal Equilibrium), PLTE (Partial Local Thermal Equilibrium states) and NLTE (Non-Local Thermal Equilibrium).

In Alzheimer Disease as shown in nMRi images, it's trivial to pay attention of the neurodegenerative process, water is lost and healthy proteins are associated to lysis of functional tissues, the degraded proteins will increase the degenerative process, with more Aβ plaques, fragments, and ROS entropy, prions and loss of constitution water (linked to tissues), and losses in tissues structures lysis (gray-white matter); FIG. 2.

The more important mission is to hydrate the brain at the standard level (balance), with the possible help of protocols SiHR57—the water level in tissues can easily determine by NMR MRI imaging with informatic adapted programs-SiHR57 can contains Na⁺, K⁺, Li⁺ (formula of Silicon Compound [3(CH₃Si(OH)₃—R(COO X)], X might be Na—K—Li).

Mode of administration of the composition: oral, N2B (nose to brain), intranasal sprays (aerosols), dermic probes, trans-dermal patches, intravenous, intraperitoneally, intracranial. Active molecules administered by means of an intracranial probe are conveyed by means of a controlled-rate syringe, the conduit of the syringe is subjected to permanent fields B around or solenoids in a pulsed or oscillating mode whose intensity varies from 0.05 T Tesla at 0.3 T Tesla (the Tesla value can be changed regarding the stage of the pathology and imagery control 1.5 T-3 T). This stimulation allows a preliminary alignment of the spins (atoms) and/or active ions.

Haemoglobin molecules, cyclodextrins, ursodeoxycholic acid, Zn-FLNT4 and part of depolymerized Aβ all under spin alignments, are subject to MHD-MGD properties.

Removal by circulation-diffusion (when dissolved) of toxic molecules (plaques, tangles and prions and cholesterol, hemosiderin, Copper Oxide, others) as a result of their dissolution and elimination via external route at BBB-by the mean of solvents under magnetic B vectors MHD-MGD protons in membranes (transportation of toxic molecules and ions out of the BBB and brain tissues) and RF-EM collateral vector.

Reconstruction protocols of neuronal tissue: use of pluripotent stem cells (stem cells) allows the future transformation of astrocytes into new neurons, the reconstruction protocol can be engaged only after the treatment of toxic molecules, fragments and plaques, protocol SiHR57 01-02.

The potential for reconstruction using astrocytes and/or glial (glial cells) under the influence of stem cells and SiHR57 protocols is considerable, 5.10¹² glial cells compared with 109 neurons. We can liken a neuron to a hard disk that can no longer retrocede memory files for lack of correct passwords (neuro-chemical and elementary mediators (metals and metalloids)) and steady radiation (UV) and magnetic fields vectors are to be considered as the observables and solutions of the Hamiltonian matrix. The dopaminergic reconstruction is therefore complex but understandable, it seems that the nuclei memory extra-nuclei of neuron is associated with an electromagnetic memory.

All these chemical and biological considerations make it possible to construct a coherent set of drug vectors adapted to particular phases of the pathologies (see the protocols and combinations of formulas SiliconHR57), but also to better understand the chemical and molecular vector associations and physical (MGD-MHD, EM radiation, UV/Lasers, pulsed magnetic fields B). The associated or separate use of these vectors makes it possible to treat the origins, causes and consequences of these molecular pathologies.

In pharmacodynamics and pharmacokinetics approaches, the important notions of magneto-susceptibility (complex molecules and natural elements) that make up future agents or therapeutic vectors are not sufficiently considered at today. This vision and consideration of the magneto-susceptibility affects the entire biological environment, which is not static, because biology is living, but our tools of investigation are somewhat insufficient for the moment.

The water molecule plays a preponderant role in all the biological and biophysical phenomena of which the MHD-MGD presents within membranes including in synaptic clefts, ion transport and ion solvation. Water in our biology mainly in membranes could be assimilated as H₂O monomer, because could adopt different degrees of polymerization n(H₂O) due to the presence of weak UV radiation in biological membranes (5-10-50 nm). We can consider water as the first quantum liquid and hold a quantum, but also comparable to a gas mM 18 and subject to MHD-MGD principles under precise conditions in the context of Hamiltonian systems.

MHD-MGD mechanisms associated with the solvation of ions H₃O⁺ protonation of the medium create kinetics that oppose the residual entropy, especially in the aggregates of Aβ proteins, APOE, within the synaptic medium, to the extent that it can be considered analogous to fibrinogen monomers and also a quasi-polymerized form.

In the problems at hand, it is considered that the protective and/or therapeutic vectors must possess a powerful action of dissolution of toxic compounds molecules and part of molecules (plaques, fibrinogen, prions, fragments).

The present approach considers mechanisms of dissolution and elimination of toxic proteins and collateral products engaged in ROS, cholesterol LDL bound to others molecules, hemosiderin-copper (prions, fragments, PrP-plaques-tangles Aβ-APOEε4-tau proteins, self-assembly or polymerized structures and degraded form) and at final the reconstruction of neural brain organization with astrocytes and master stem cells—methods and protocols SiliconHR57-SiHR57 (01 to-04) and strategies under RF-EM pulsed electromagnetic fields and permanent fields or solenoids (pulsed)—and under NMR 1.5 to 3 T- and permanent 0.050 T to 0.1 T or pulsed or oscillating field Ferromagnetic species metals and Lanthanides as metals T values can be changed and adapted to several routes for the drug SiHR57-endonasal N2B-intracranial-.intravenous-oral.

The present approach considers the use of Zn-FLNT4 cyclic formula Zn associated with D-Fructose molecule on C═O Carbonyl Radical(patent) (used as solvent) ZnFLNT4 (cyclic molecule) associated to Zinc (patent) as main solvent of toxic molecules associated to the degenerative process engaged in AD, Aβ plaques and prion fragments, Tau proteins (self-assembly CHO and/or polymerized structures and degraded form), APOEε[4], hemosiderin degraded. (ZnFLNT4) cyclic Electrochemistry process—Zn concentration 400 mg/L to 8000 mg/L used in protocols. Si element concentration for Liquids 300 mg/L to 3500 mg/L and for gels 20′000 mg/L used in protocols.

The present approach considers the use of silicon compound (3(CH₃Si(OH)₃—R(COO X)) wherein X can be Na—K—Li (hepato-neuro protector). Germanium or other tetravalent element as well as SULFUR S32 can be used in place of Silicon or Carbon or associated with the Silicon composition.

The present approach considers the use of cyclodextrin(s) associated to one or several Zn atoms (av 65.4 mass) forming the cage molecule of Cyclodextrin(s) (α 972-β 1135-γ 1297), wherein the most soluble in water is γ CD 23%. The cyclodextrin cage associated with Zn is elaborated with an electrochemical process in the presence of Zinc, D-Fructose and also can be prepared with labeled Zinc 70. The external surface of cyclodextrins, in cage or torus shape, can accept hydrophilic compounds as well as Zn Carbonyl or others natural elements. Silicon-germanium nano or pico particles, or other natural elements (precious metals and the like, hydrophobic compounds) can be lodged on the inner surface of cyclodextrins, whether in cage or torus shape, whereby cyclodextrins can be considered nano-carriers or pico-carriers. Cyclodextrins are under MHD-MGD process—BBB under MHD ions) and MGD process(p+-(H⁺)) membranes (5 nm-8 nm). Synaptic cleft is under MHD-ions Ca²⁺ (others) Zn²⁺ (mediators) and under MGD process H⁺ membranes of high velocities. 10⁶ ions/s−1. Astrocytes are under influence of MHD-MGD process and contribute to the standard DDP (difference of potential) in the BBB layers and membranes.

The process of the release of natural elements on the elected area (neuronal tissues) is quite complex, we can consider that the Cyclodextrins cage or torus in general will obey at MHD-MGD observables—in all cases we can establish the Hamiltonians H (Harmonic Oscillator) and observables engaged in the quantum response under (radiative vectors and EM RF vectors), takes place in the Quantum Discussion. In these specialized cases of polarization and depolarization the matrix will obey to the creation a′ and a annihilation operators (quantum mechanisms formalism). The polarization effect and depolarization effect will conduct assume the binding and the release of the future active substances. RF EM selected waves length and frequencies will organize and will engage and leads to the “drug” delivery consequence.

The present approach considers the use of cyclodextrin(s) cages in presence of Ln³⁺ (lanthanides or Rare Earths) elaborated with electrochemical process in situ CD and (Ln³⁺/La-Nd-Lu among others/14 and Y(Yttrium and/or Sc) for NMR-fMRI imaging with the labelled element of Ln³⁺, Tc⁺³ (99m) Technetium or Gadolinium Gd³⁺ (in the NMR imaging (Neuro imaging and Cardiovascular coupling) while the drug SiHR57 is distributed in the via the (BBB) or others routes, the results are obtained online coupled (patient under NMR fMRI and under efficient drug)—the monitoring will be considered as powerful too in order to establish the best routes for the drug and the choice of doses and protocol (adapted to the grade or stage of the pathology) AD and CVD Cardio Vascular Disease with the preliminary stage of Cardio Vascular Inflammation, and neuro immune deficiency, route N2B seems to be the more interesting clinical and investigation method. The concentration of cyclodextrins will be different depending the route of the drug and the solubility chosen SiHR57, 30 mg to 3000 mg. Lanthanides (Rare Earths) can be prepared under MHD-MGD tools.

The present approach considers the use of experimental physics concepts, such as the application of a MHD-MGD system to compositions, compounds and/or biological molecules or chemicals vectors, with the capabilities to constitute a cage shape, potentially as a toroidal construct, with aliphatic and/or aromatic compounds. MHD-MGD can be applied to cyclodextrins-oses, polyoses-phenols and polyphenols, molecules of hemoglobin (and BBB as physical shape (tube or sphere envelope) with heme structures circulating, creating a magnetic field outside of BBB by Fe⁺² displacement of iron ions inside the BBB efflux. Glycans, proteoglycans and glycoproteins can be submitted under MHD-MGD process. The present approach considers the use of the silicon compositions described herein as nano-particles (FIGS. 13, 14 and 15 MGD-MHD).

With an average molecular weight of 94 mⁱ to 134 m^{j}, SiHR57 molecules according to the formula (3(CH₃Si(OH)₃—R(COO X)) (with X as one of Na, K or Li) can easily pass through the BBB into CNS. This property is considered to explain the results obtained in both in vitro and in vivo tests performed by the Faculty of Pharmacology of the University Complutense of Madrid (UCM) with SILICON G57 in 2015.

The present approach considers the use of the plasma-based MHD-MGD process (FIGS. 13, 14, 15) disclosed in US20180073361A1 for manufacturing nanoparticles and picoparticles, which is hereby incorporated by reference. In the case of picoparticles, the most elegant approach relies in using a Magneto Hydro Dynamics (MGD) process, with natural elements (metals or metalloids) introduced as plasma seeds into the plasma tools. And recycled MHD-MGD 5000 to 14000 cycles in the toroidal machine, after the thermal and magnetic treatment, we obtain a true metal “solution” of metals inside plasma gases as well in liquids—the wording [solution] can be considered as justified if we consider the plasma state under MHD-MGD with properties of quantum liquid with radiative properties in place of water-water mw=18 can adopt different state of matter, some authors mainly astrophysicists consider water as the first quantum liquid. Under precise conditions we can apply the laws of electrochemistry (water or liquids) but also in quantum gases. This new route could be of interest for the elaboration of Nano P crystals for pharmacology Earth and Space, and to quantum crystals devices mainly with Lanthanides and Actinides stabilized with rare gases included isotopic species as well as ³He₂ (others rare gases included Rn222) and in order to trap radioactive isotopes for further radio decay treatments in order to build radioactive shields (space medicine and protection against solar system radiations or beyond).

This applied research will be of high interest in deep space or micro gravity laboratories. High energy radiation such as [symbol] and X rays, and other particles from solar activity [symbol] and from deep space origin can easily interfere with DNA-RNA-nucleic bases and telomers and car damage seriously the structure. SiHR57 protocols can be used to supply the compositions, or functional food supplements including the composition, to astronauts for short or long trips in the solar system and beyond, likewise for medium to long residence at extra-terrestrial locations, in order to avoid the neurodegenerative situations. The modification of some nucleic base with substitution of Carbon atoms with Silicon or Germanium or Sulfur atoms can create a biologic shield: the Silicon or Germanium will absorb the radiation and will generate electric current flux, we can consider this phenomenon as negentropic. The loss of electric capability in our biology and structures can be considered as entropic situation. In particular conditions the current conditions can excite Silicon/Germanium crystals and the Si and Ge will emit EM radiation mainly UV (Ultra Violet), this phenomenon in DNA at low level of energy, will help the neural activities (clefts- and BBB-CNS) and the rebuilding of neural tissues with master stem cells and astrocytes.

The use of MHD-MGD at several orbitals LEO-NEO-GTO and deep space conditions is completely favorable due of the natural existing high vacuum 10⁻¹³ to 10⁻¹⁷ torr confirm the interest to engage experiments in Orbital Spatial Stations near Earth or Moon(s). The Nano P field can be used in space medicine (micro gravity) orbital Spatial Station or Nano, Pico or Femto gravity (deep space) and precisely for cancer research and DNA-telomers rebuilding. In addition, the Nano-P program can be associated to SN1-SN2 CRISPER Cas9 gene editing (sequencing) in order to protect DNA and telomers from solar radiation or cosmic radiation.

The species Nano-P and Pico-P is using a CD(s) carrier and FLNT4 patent (d-fructose) or other organic compounds and other metals. The NANO P are trapped in the Cyclodextrins cage with the help of EM waves field of polarization selected by the lab, the release of the drug will be engaged with depolarization EM waves field—while the destination of the drug is verified by NMRi imaging. The elaboration of [NANO-PICO]P will be utilized in order to rebuilding the DNA proteins- and telomers—by sequencing engineering CRISPER cas9. The protocols might be used in the chelation of low-radioactive compounds by molecular cages among others Cyclodextrins.

NanoP are fully active if the ROS has been decreased at the lowest level, the protocol SiHR57 can be associated to Nano P treatment, mainly in cancerology (brain tumors astrocytomes). The protocols SiHR57 can be associated with others protocols as well radiotherapy and immunotherapies. Cyclodextrins can be used in electrochemical processes as described in WO/1996/023089, which is hereby incorporated by reference.

The present approach considers the use of the SiHR57 compositions, protocols and the pathologic molecules compounds and plasma tools described in the protocols for preparing new tools in biologic analysis including a quantum electro magnetophoretic apparatus intended for research or routine biological analysis. A known tool includes an electrophoretic apparatus with two-dimensional spectral image (film) or plate or screen of proteins displayed, the new tool develops 3D spectral image (animation), included the affected area and will avoid errors or uncertainties on results.

The molecules are analyzed under magnetic fields B (permanent-pulsed)/EM field and provide spectral analysis (magnetic gradient of response). This quantum tool is different from NMR analysis laboratory tool, the concerned molecules are spread on plates which are submitted to B-EM vectors, results are delivered in 3D images and not in 2 D images as well known in the classical electrophoretic analysis. The quantum tool will be able to detect the default of the molecular structure of proteins and part of proteins and will instruct the biologist where to use drugs or genetic tools (gene sequencing)—valid molecules under magnetic field deliver quantum calibrated responses (health value-opposed to unknown or pathological values).

Evaluation of the neuroprotective activity of Silicon HR57 within an active composition is conducted through in vitro and in vivo tests.

For in vitro tests, the Acetylcholinesterase (AChE) activity is determined spectrophotometrically by the Ellman method (Ellman et al., 1961) modified by Froede and Wilson (1984).

β-secretase activity is carried out by cleavage in Leu-Asp of a specific peptide for the β-secretase to finally produce a fluorescent signal, which is proportional to the degree of hydrolysis of the peptide substrate, and therefore, to the enzymatic β-secretase activity. Recombinant human β-secretase (Sigma-Aldrich) and a fluorogenic substrate is used. Fluorescence is measured using a fluorescence reader for multi-well plates (Ni et al., 2013).

The determination of γ-secretase activity is based on the excision of a specific peptide for the γ-secretase to produce a fluorescent signal. The intensity of the fluorimetric signal is proportional to the degree of hydrolysis of the peptide substrate in Val-Ile-Ala, and therefore, to the γ-secretase enzyme activity. The aspartyl protease complex of γ-secretase is prepared from the membrane isolation of APPswe cells (Esler et al., 2002).

For in vitro fibrillogenesis of β-amyloid peptide, it has been proposed that the Aß peptide (25-35) represents the biologically active region of peptide 1-42, since it is the shortest fragment showing a conformational transition from a soluble form to an aggregated fibrilar form consisting of β-sheet structures and, that in addition maintains the toxicity of the full-length peptide (Pike et al., 1995). For this reason, the fragment 25-35 is used in this study. A simplified model of in vitro fibrillogenesis is established from a commercial peptide (25-35) in the monomeric form in solution that is incubated at 37° C. in a humid atmosphere for a period of 96 hours and with constant agitation (Stine et al., 2003). The process of in vitro fibrillogenesis under the influence of SilHR57 is both qualitatively and quantitatively evaluated.

Qualitative evaluation is carried out by transmission electron microscopy. Quantitative evaluation uses a fluorimetric test of both the stationary and the resolved phases. The determination of the lifetimes allows the identification of potential inhibitors of the βA peptide aggregation and/or disaggregation. The variation in the fluorescence half-life due to the presence of SiHR57 allows quantification both of the inhibition of the βA peptide aggregation and of its disaggregation level.

For in vivo tests, testing SiHR57 in the APPswe cell line allows for the selection of a non-toxic concentration range of this product (Mosmann, 1983); for the examination of the potential neuroprotective effect of SiHR57 by determining the inhibition of free radicals generated from exposing the cells to hydrogen peroxide (Garcimartin et al., 2014).

Next is the BV2 cell line, derived from murine neonatal microglia, which is frequently used as an alternative of primary microglia, because it has certain advantages related to difficulties in maintaining adequate cell culture and functionality (Blasi et al., 1990). This cell line helps determine how SiHR57 modifies nitrite release after activation with interferon INFγ (Chow et al., 2012). The determination of nitrites, as stable metabolites of NO, is carried out in BV2 cells activated with interferon INFγ. The quantification of these metabolites is carried out using the colorimetric method based on the Griess reaction. This reaction is based on the formation of a chromophore by the reaction of sulfanilamide with nitrite in an acid medium, followed by a coupling. NO-2 reacts with sulfanilamide in an acid medium producing a diazo compound, which subsequently, through a coupling reaction with N-1-(naphthyl) ethylenediamine, gives rise to a purple azo compound. 2) Neuroprotective effect of SILICON HR57 against inflammation stimulated by LPS in microglial cells BV2 and determination of free radicals (Cai et al., 2018).

By way of statistical analysis, data is processed using the statistical package SPSS (statistical package for social sciences), version 25.0. The mean and standard error of the individual values obtained for the samples of the animals of each group is determined. Normality distribution of data is determined, using parametric or nonparametric test when data normalization would be unavailable. The analysis of variance (ANOVA) or the multiple comparison test of Kruskal Wallis, followed by the adequate post-hoc test, is used to compare values obtained from different groups and treatment. A multivariant test (e.g. MANOVA) is also applied, wherein P values <0.05 is considered significant.

The SiHR57 study is initiated in a model of experimental Alzheimer's. The mouse transgenic line Tg2576 (B6; SJL-Tg (APPSWE) 2576 Kha) is selected, heterozygous “knock-in” line, which carries the hAPP with the Swedish double mutation of Alzheimer's disease (K670N, M671L) that favours the action of the [symbol] -secretase instead of the α-secretase, giving rise to accumulations of Aß in the brain, either diffusely or forming plaques. These deposits begin approximately at 9-12 months of age of the animals. They also present cognitive deficits, learning and spatial memory, damage by free radical action, oxidative stress and apoptosis.

The in vivo tests aim to evaluate the general motor activity of Tg2576 mice at 18 months of age, after having been chronically exposed to a dose of an aggressive agent (eg 1 mg Aluminum/g of diet and 3.2% of citric acid) and the effect of silicon HR57 compositions thereon, and to evaluate whether silicon HR57 compositions can modify the concentration of Aβ in the cortex and hippocampus of Tg2576 mice.

The total number of animals at the beginning of the study is at least 100 (50 wild-type and 50 Tg), with genotyping at 4 weeks. The number of deaths stipulated by the house is 33%.

Reagents used for in vivo experiments include: for the treatment of animals, standard maintenance diet (Panlab)., diet supplemented with aluminum lactate and citric acid (1 mg Al/g of diet and 32 g of citric acid/Kg of diet) (Harlan); for genotyping, Tris Base (2-Amino-2-hydroxymethyl-propane-1,3-diol) (Sigma), EDTA (Sigma), SDS (Sodium dodecyl sulfate) (Sigma), Proteinase K (Sigma) and sodium chloride ethanol; for the sacrifice of animals, Ketamine hydrochloride, 100 mg/ml, Xylazine hydrochloride 20 mg/ml; for the determination of accumulated Aß, AEBSF (4-(2-aminoethyl)-benzenesulfonyl fluoride) (Sigma), Guanidine hydrochloride (Sigma), Potassium chloride (KCl) (Sigma), Basic sodium phosphate (Na2HPO4) (Sigma), Acid potassium phosphate (KH2PO4) (Sigma), Sodium hydrogencarbonate (NaHCO₃) (Merck), Human AR40 ELISA kit KMB3481 (Invitrogen), Human AR42 ELISA kit KMB3441 (Invitrogen), Bradford reagent (Sigma), Bovine serum albumin (BSA) (Sigma).

Devices or apparatuses used for in vivo experiments include: for genotyping, a source of electrophoresis, a temocycler, an Image Master VDS; for behavioral tests, an open field (OF), a Morris Water maze, a video camera and computer software. For the detection and analysis of the movement of animals in the OF and in the Water maze, the video camera is coupled to a computer configured for use with the the Etho-Vision program; for the determination of Aß fragments, a spectrophotometer plate, an ELISA kit for Aβ(1-40), Aβ(1-42) and Aβ(25-35).

The compound and techniques described above find further application in respect of pathologies arising from contamination by the COVID-19 virus. We can consider the biodegradation of pulmonary embolism and toxic molecules produced similar to (CVA) Cardio Vascular Accident and Ischemic Stroke (AIS): in the case of COVID 19, the toxic molecules are among others hemosiderin and bio-degraded fragments of the COVID 19 genome sequence. Dechelation agents can be used, and mainly applied to hemosiderin with deferiprone (DFP), desferioxamine (DFO) and deferasirox (DFX) compounds or heparine.

Hemosiderin is the logical molecular consequence of the biodegradation of haemoglobin. Hemosiderin constitutes the main barrier to oxygen exchanges in the pulmonary alveolar functions and creates clots, blocking the oxygen chain supply to eventually cause patient death. We can consider the pathologic results in the PAP and PE as the direct action of the viruses involved in reactive oxidative stress (ROS), causing strong oxidation and peroxidation of the blood compounds and thus generating the hemosiderin, among others.

We can logically conclude that this oxidative stress produces polymers of cholesterol and protein plaques similarly to Alzheimer's Disease, thus it is logical to consider use of the DFP-DFO-DFX chemical compound in the Alzheimer Disease protocol, for solubilizing bio-degraded hemosiderin compounds.

In terms of procedure, the patient should undergo the whole lungs lavage (WLL) protocol as described in “Whole lung lavage-technical details, challenges and management of complications” by Ahmed Awab, Muhammad S. Khan, and Houssein A. Youness in J Thorac Dis. 2017 June; 9(6): 1697-1706 (doi: 10.21037/jtd.2017.04.10, PMCID: PMC5506114, PMID: 28740686), pursuant to a medicinal decision based on a double opinion, after a biological survey and control parameters have been established to determine how LL should be applied to the patient, including any counter indications against DFP-DFO-DFX for the patient under these molecules.

A biologist should preferably collect a patient sample before, during and after WLL treatment, to determine and then monitor bio-chemical compatibility and toxicity of the drugs, together with the correct dosage according to the specific patient and their admission status at either intensive care or reanimation stage. Several CT scans of the patient at pulmonary level should preferably also be carried out at least before and after the WLL procedure, but also during the WLL procedure if resources allow.

Additional active compounds may be used either during the WLL procedure, or thereafter, in addition to the DFP-DFO-DFX compound, such as HydroxyChloroQuine (HCQ), antibiotics (notably azithromycin among as described in international patent application WO1996/023089 A1) as well as zinc salts such as Zinc citrate (ZnFLTNT4), a Zn-Carbonyl compound and ortho-silicic-acid-or-3 (CH₃Si(OH₃)Xₙ[CH₂—COONa]₃ in order to protect alveolar functions.

The invention is not limited to the embodiments hereinbefore described but may be varied in both construction and detail. In the specification the terms “comprise, comprises, comprised and comprising” or any variation thereof and the terms include, includes, included and including” or any variation thereof are considered to be totally interchangeable and they should all be afforded the widest possible interpretation and reciprocally.

There follows a list of acronyms used in the present disclosure for ease of reference:

| | |
|---|---|
| H: | Hamiltonian |
| a [symbol] : | creation operator |
| a: | annihilation operator |
| AD: | Alzheimer's Disease |
| CVA: | Cardio Vascular Accident |
| Zn FLT4 | cyclic(Zinc Carbonyl) |
| CAA: | Cerebral Amyloid Angiopathy |
| CDs (αβγ): | cyclodextrins ursodeoxycholic acid |
| UR: | ursodeoxycholic acid |
| EM-RF: | electro magnetic radio waves |
| APOEε[4]: | apoliprotein E |
| E-Aβ: | amyloid protein |
| Aβ(1-42)- | prion framents |
| (1-40)-(25-35) | |
| PrP: | prion protein fragments |
| NMR: | Nuclear Magnetic Resonance |
| MHD: | Magneto Hydro Dynamics |
| MGD: | Magneto Gas Dynamics |
| BBB | Blood Brain Barrier |
| Fe+2: | iron ion+2 ionic state |
| Fe+3: | iron ion+3 ionic state |
| FeII: | iron element II ionized state |
| FeIII: | iron element III ionized state |
| Hemoglobin mRNA: | Hemoglobin messenger RiboNucleicAcid |
| DNA: | DeoxyriboNucleic Acid |
| LTE: | Local Thermal Equilibrium |
| NLTE: | Non Local Thermal Equilibrium |
| PLTE: | Partial Local Thermal Equilibrium |
| | (plasma physics) |
| NP: | Nano Particles |
| PP: | Pico Particles |
| EM list: | list of electromagnetic (frequencies) |
| | as a matrix (elaboration of the protocols) |
| | part of the Hamiltonian configuration |
| T: | Tesla value of the field |
| N2B: | Nose to Brain route |
| CNS: | Central Nervous System |
| CHO (LDL-HDL): | Cholesterol monomer molecules |
| m [symbol] : | molecular weight |
| PMGs: | Precious Metal Group |
| ROS: | Reactive Oxygen Species |
| UCM | University Complutense of Madrid |
| Silicon G57 | first generation of Silicon compound 2014 |
| PCBs: | PolyChlorinated Biphenyl |
| S32: | Sulfur element (average atomic mass) |
| MMM: | MonoMethyl Mercury |
| RPE-SRE: | Spin Orbit coupling |
| GC-MS-LS techniques: | Mass spectrometry techniques |
| NMRi(NMRI) : | Nuclear Magnetic Resonance Imaging |
| NFT: | Neuro Fibrillary tangles |
| LEO: | Low Earth Orbital |
| NEO: | Near Earth Orbital Object |
| GTO: | Geostationary Transfer Orbit |
| SN1-SN2: | Substitutions Nucleophylic protocol |
| CRISPER Cas 9: | gene editing(sequencing) technology |

1. A composition comprising zinc Zn-FLNT4 in the form of an organic compound soluble in water and having a carbonyl function complexed with zinc, and silicon SiHR57 in the form of (3(CH₃Si(OH)₃—R(COO x)), wherein x is selected from the group comprising sodium, potassium and lithium, for use as a medicament or in therapy.

2. The composition according to claim 1, wherein Zn-FLNT4 is associated with a D-fructose molecule.

3. The composition according to claim 1, wherein the organic compound soluble in water and having a carbonyl function is at least one selected from a sugar, an oligosaccharide, a disaccharide, an enzyme, a coenzyme, a protein, a peptide, an amino acid, a hormone, a steroid, a prostaglandin, a neurotransmitter, a vitamin, an ester, a carboxylic acid, a ketone, an anhydride, and an antibiotic.

4. The composition according to claim 1, for use as a medicament or in therapy, in combination with a whole lung lavage procedure.

5. The composition according to claim 1, for use in a method for the treatment of neurodegenerative and cardiovascular diseases.

6. The composition according to claim 5, wherein the method further comprises the use of a third composition comprising cyclodextrins (αβγ).

7. The composition according to claim 5, wherein the method further comprises the use of a fourth composition comprising ursodeoxycholic acid, optionally the method further comprises the use of master stem cells.

8. The composition according to claim 1, for use in a method for mitigating neurodegeneration associated with exposure to at least one selected from solar energetic particles, galactic cosmic rays and extra-terrestrial radiation.

9. The composition according to claim 8, incorporated into one or more human food supplement(s) for delivery.

10. A method of aligning the spin of molecules in the composition according to claim 1, comprising the steps of:
locating at least one conduit adjacent a source of electromagnetic radio waves;
causing the composition to flow through the conduit; and
generating electromagnetic radio waves with the source, for subjecting composition in the conduit to the generated electromagnetic radio waves.

11. The method according to claim 10, wherein the step of generating further comprises generating the electromagnetic radio waves with intensity in the range 0.05 Tesla to 0.3 Tesla, or in the range of 1.5 Tesla to 3 Tesla.

12. The method according to claim 11, wherein the step of generating further comprises generating the electromagnetic radio waves as permanent electromagnetic radio waves, pulse-modulated electromagnetic radio waves or oscillating electromagnetic radio waves.

13. The method according to claim 10, comprising the further steps of
administering the electromagnetic radio waves-subjected composition to the patient;
locating at least an area of the patient including the administered composition adjacent a magnetic resonance imaging apparatus; and
generating second electromagnetic radio waves with the magnetic resonance imaging apparatus, for subjecting the area including the administered composition to the generated second electromagnetic radio waves.

14. The method according to claim 13, wherein the step of generating the second electromagnetic radio waves further comprises generating the second electromagnetic radio waves with intensity in the range 1.5 Tesla to 3 Tesla.

15. The method according to claim 10, wherein the step of administering is performed through one selected from oral ingestion, endonasal or intranasal spray, dermal probe, transdermal patch and intraperitoneal or intracranial injection.

16. The method according to claim 10, wherein the conduit is part of a controlled-rate syringe.

17. A cyclodextrin particle for delivering the composition according to claim 1.

18. The cyclodextrin particle according to claim 17, wherein the particle is shaped as a three dimensional cage with at least one aperture, or is shaped as a torus.

19. The cyclodextrin particle according to claim 17, further comprising silicon-germanium nanoparticles or picoparticles located on an inner surface of the cyclodextrin particle.

20. The cyclodextrin particle according to claim 17, further comprising Zinc carbonyl located on an outer surface of the cyclodextrin particle.
